(19) United States
(12) Patent Application Publication
(10) Pub. No.: US 2022/0331357 A1
(43) Pub. Date: Oct. 20, 2022

(54) COMPOSITIONS AND METHODS FOR THE PREVENTION AND TREATMENT OF PANCREATITIS

(71) Applicant: Duke University, Durham, NC (US)

(72) Inventors: Rodger LIDDLE, Durham, NC (US); Ahmad FAROOQ, Durham, NC (US)

(21) Appl. No.: 17/763,740

(22) PCT Filed: Sep. 28, 2020
(86) PCT No.: PCT/US20/53024

§ 371 Date: Mar. 25, 2022

Related U.S. Application Data
(60) Provisional application No. 62/906,870, filed on Sep. 27, 2019.

(51) Int. Cl. A61K 33/42; A61K 33/06; A61K 45/06; A61K 31/444; A61P 1/18

(52) U.S. Cl. CPC A61K 33/42; A61K 33/06; A61K 45/06; A61K 31/444; A61P 1/18

(57) ABSTRACT

The present disclosure provides compositions comprising sodium phosphate for the prevention and treatment of pancreatitis and methods of using same.

### CROSS REFERENCE TO RELATED APPLICATIONS

This application is a 35 USC 371 application of International PCT Patent Application No. PCT/US2020/053024, filed on Sep. 28, 2020, which claims priority to U.S. Patent Provisional Application No. 62/906,870, filed on Sep. 27, 2019; the contents of which are hereby incorporated by reference herein in their entireties.

### FEDERAL FUNDING

This invention was made with Government support under Federal Grant Nos. DK064213 and DK125308 awarded by the NIH. The Federal Government has certain rights to this invention.

### BACKGROUND

Pancreatitis is a severe, painful, and debilitating disease which has several etiologies, and for which there is no specific treatment. Chatila AT, et al., Evaluation and management of acute pancreatitis, World J. Clin. Cases May 6; 7(9): 1006-1020. Over 200,000 patients are hospitalized in the United States each year with pancreatitis and severe acute pancreatitis is associated with a ˜20% mortality rate. Treatment of pancreatitis has proven difficult once the disease has been initiated.

Strategies to treat or prevent the development or progression of pancreatitis are of great need.

### SUMMARY

The present invention in various aspect and embodiments provides compositions and methods for preventing or treating pancreatitis. The invention in various embodiments comprises administering a composition comprising an effective amount of phosphate to the subject so as to treat or prevent pancreatitis in the subject.

In some embodiments, the subject is at risk for developing pancreatitis. In these embodiments, the patient may or may not have clinical hypophosphatemia, but phosphate supplementation will protect from organ injury. In these embodiments, the subject may be at risk of pancreatitis of any etiology. When treating a subject at risk for pancreatitis, the subject may or may not have clinical hypophosphatemia. In accordance with embodiments of the invention, phosphate treatment can be provided prophylactically before serum phosphate levels decline and before hypophosphatemia (subclinical or clinical) develops.

In some aspects, the subject presents with pancreatitis, including pancreatitis of any etiology. The subject may have chronic or acute pancreatitis. In accordance with embodiments of the invention, administration of phosphate early (e.g., by i.v.), such as to patients presenting with mild acute pancreatitis, can prevent or slow progression of the disease. As described above, a subject presenting with mild acute pancreatitis may or may not have clinical hypophosphatemia, and phosphate treatment may be given before serum phosphate levels decline and before hypophosphatemia (subclinical or clinical) develops. In some embodiments, before undergoing phosphate therapy, the subject is tested for phosphate levels, and phosphate is administered if the patient does not have hyperphosphatemia.

In some embodiments, the treatment with phosphate comprises sodium phosphate and/or potassium phosphate. For example, in some embodiments, the treatment involves intravenous administration with, for example, sodium phosphate. For example, the sodium phosphate injection can be provided as a concentrated solution containing a mixture of monobasic sodium phosphate and dibasic sodium phosphate in water for injection. In some embodiments, i.v. phosphate treatment is administered to the subject prior to or after undergoing ERCP to protect the subject from developing pancreatitis.

In some embodiments, phosphate supplementation is administered orally. For patients at risk of pancreatitis, or having chronic pancreatitis or prior episode of acute pancreatitis, oral phosphate supplementation protects from pancreatitis or further episodes or severity thereof. In various embodiments, the oral supplementation is provided in tablet, capsule, or liquid form, and can be administered, for example, in a daily dose of from about 300 mg to about 1500 mg.

In some embodiments, the patient is further administered one or more agents that may act additively or synergistically to protect from pancreatitis. Such agents are selected from one or more of: an alpha 7 nicotinic acetylcholine receptor agonist, TRPV4 antagonist, an anticoagulant, pancreatic enzyme inhibitor, magnesium, non-steroidal anti-inflammatory drug (NSAID), calcineurin inhibitor, and calcium channel blocker. Such agents may be administered in the same composition as the phosphate (together with one or more pharmaceutically acceptable carriers or excipients) or separate composition. In various embodiments, such agents are administered by i.v. or orally, or by other appropriate route.

In still other aspect, the invention provides a pharmaceutical composition comprising an effective amount of a phosphate salt (such as a sodium and/or potassium phosphate salt) and one or more of: an α7 nicotinic acetylcholine receptor agonist, TRPV4 antagonist, an anticoagulant, pancreatic enzyme inhibitor, magnesium, non-steroidal anti-inflammatory drug (NSAID), calcineurin inhibitor, and calcium channel blocker. Such compositions can be formulated for any administration route, including intravenous administration, intradermal or transdermal administration, and oral administration. These compositions can provide novel formulations for treating or preventing pancreatitis, and may have additive or synergistic properties for treating or preventing pancreatitis, as compared to the agents individually.

### BRIEF DESCRIPTION OF THE DRAWINGS

The various aspects and embodiments of the disclosure are explained in the following description, taken in connection with the accompanying drawings.

FIG. 1 shows a model of how phosphate supplementation may rescue pancreatic injury by stabilizing mitochondrial function and ATP production in pancreatic acinar cells.

FIGS. 2A and 2B shows that caerulein pancreatitis causes hypophosphatemia in accordance with one embodiment of the present disclosure. Wild type mice were treated with 6 hourly injections of caerulein (50 ng/g). Serum phosphate levels were measured 7 hours after the initial caerulein injection (A). Serum amylase and pancreatic MPO levels (A) and histology (B) were also evaluated 7 hours after the initial caerulein injection. **P<0.01 vs. control. Control group n=4 and caerulein group n=5.

FIGS. 3A-3C show that sodium phosphate treatment ameliorates pancreatitis in accordance with one embodiment of the present disclosure. Mice were treated with 6 hourly injections of caerulein. Sodium phosphate (Na₂HPO₄) and normal saline (NS) were given 1 hour after the first injection. Pancreatic myeloperoxidase (MPO) (B) and serum amylase (A) levels were measured by commercially available assays. FIG. 3C shows tissue histology. **P<0.01 vs. normal saline in caerulein-treated mice.

FIG. 4 shows effects of phosphate on caerulein-induced injury and reduction of cytosolic ATP levels in isolated pancreatic acini in accordance with one embodiment of the present disclosure. Isolated mouse pancreatic acini were treated with caerulein in buffer containing Na₂HPO₄ concentrations of 1-10 mM. Supernatant LDH was measured by commercial LDH assay (left panel). Cytosolic ATP levels were measured using a commercially available fluorometric assay (right panel). *P<0.05, **<0.01, ***<0.001.

FIGS. 5A-5C show that phosphate protects against caerulein-induced damage in isolated pancreatic acini in accordance with one embodiment of the present disclosure. Levels of trypsin (FIG. 5A), cytosolic IL-1β (FIG. 5B), and LDH (FIG. 5C) were evaluated. *P<0.05, **P<0.01, ***P<0.001.

FIG. 6 shows that caerulein causes mitochondrial membrane depolarization (left) and reduces ATP in pancreatic acini (right) in accordance with one embodiment of the present disclosure.

FIG. 7 demonstrates that phosphate restores mitochondrial function (left) and ATP production in pancreatic acini (right) in accordance with one embodiment of the present disclosure.

FIGS. 8A and 8B show that hypophosphatemia exacerbates pancreatitis in accordance with one embodiment of the present disclosure. To induce hypophosphatemia, mice were placed on a low phosphate diet (LPD) for 3 weeks. ERCP pancreatitis was then induced in control mice on a normal diet (ND) and hypophosphatemic mice on a low phosphate diet (LPD). Sodium phosphate (200 μg/g) or normal saline (NS) was given by intraperitoneal injection 1 and 4 hours after induction of ERCP pancreatitis. ***P<0.001 vs. normal diet. FIG. 8A is a survival plot. FIG. 8B quantifies pancreatic edema.

FIG. 9 shows that low phosphate diet predisposes to more severe pancreatitis in the ERCP-induced pancreatitis model in accordance with one embodiment of the present disclosure. Pancreatitis parameters are quantified. *P<0.05, **P<0.01, ***P<0.001, ****P<0.0001.

FIG. 10 shows that phosphate ameliorates ERCP-induced pancreatitis in accordance with one embodiment of the present disclosure. Animals were treated with sodium phosphate (NaH₂PO₄ 50 mg/kg) by IP injection 2 and 4 hours after surgery. FIG. 10 shows pancreatitis parameters and pancreatic histology measured 24 hrs after induction of pancreatitis. *P<0.05, ***P<0.001, ****P<0.001 (n=3-8).

FIG. 11 shows that shear pressure causes sustained elevation in [Ca2+], mitochondrial depolarization (A, B), and trypsin activation (C) in pancreatic acinar cells in accordance with one embodiment of the present disclosure.

FIG. 12 shows serum phosphate levels in mice fed a low phosphate diet for 3 weeks (****P<0.0001) in accordance with one embodiment of the present disclosure.

FIG. 13 shows that low phosphate diet renders mice susceptible to ethanol pancreatitis in accordance with one embodiment of the present disclosure. Serum amylase levels, pancreatic edema, and histological scoring for pancreatitis severity are shown. *P<0.05,***P<0.001, ****P<0.0001. Bottom panels: representative histological H&E sections are shown for each condition.

FIGS. 14A-4D show that phosphate treatment protects mice from ethanol-induced pancreatitis in accordance with one embodiment of the present disclosure. Mice were fed a low phosphate diet for 3 weeks and then gavaged with ethanol (3 mg/kg) daily for 5 days. In the treatment group, mice were administered sodium phosphate (50 mg/kg) by orogastric gavage daily for 5 days. Twelve hours after the last ethanol dose, animals were euthanized. Serum amylase levels (A), pancreatic edema (B), and histological scoring for pancreatitis severity (C) are shown (*P<0.05, ****P<0.0001). The survival of mice in this study is shown in FIG. 14D. Phosphate administration significantly reduced mortality of ethanol-treated mice on the low phosphate diet (*P<0.0001, n=17).

FIG. 15 shows that ethanol-induced acinar cell dysfunction was modified by phosphate levels in accordance with one embodiment of the present disclosure. (A) Acini from C57BL/6J mice maintained on a normal diet (ND) or a low phosphate diet (LPD) were pooled from 1-3 mice and incubated with 0-50 mM ethanol in phosphate-free buffer. (B) LDH release and (C) trypsin activity were assessed. Pooled acini from LPD-fed mice were then prepared in phosphate-free buffer and treated with 50 mM ethanol and 0-5 mM Na₂HPO₄. (D) LDH release and (E) trypsin activity were measured. Fold change represents the results of test groups compared to the control condition (no addition of ethanol or phosphate).

FIG. 16 shows that phosphate level influenced acinar cell susceptibility to ethanol-induced injury by altering cellular ATP stores and mitochondrial function in accordance with one embodiment of the present disclosure. Pooled acini from C57BL/6J mice maintained on a normal diet (ND) or a low phosphate diet (LPD) were prepared in phosphate-free buffer and treated with 0-50 mM ethanol. (A) Total cellular ATP content and (B) mitochondrial membrane potential (TMRE fluorescence) were measured. 20 μM FCCP, a mitochondrial uncoupler, was used as a control. Pooled acini from LPD-fed mice were then isolated in phosphate-free buffer and treated with 50 mM ethanol and 0-5 mM supplemental Na₂HPO₄. (C) Total cellular ATP content and d mitochondrial membrane potential (TMRE fluorescence) were measured. Fold change represents the results of test groups compared to the control condition (no addition of test agents). *P≤0.05; **P≤0.01; ***P≤50.001; ****P≤0.0001. (n=3).

FIG. 17 shows that nicotine dose-dependently inhibits ERCP pressure-induced pancreatic inflammation, including pancreatic edema, serum amylase, pancreatic MPO levels, and histopathology in accordance with one embodiment of the present disclosure. **P<0.01 vs. Pressure −/Nicotine 0; ***P<0.001 vs. Pressure −/Nicotine 0; ****P<0.0001 vs. Pressure −/Nicotine 0; #P<0.05 vs. Pressure +/Nicotine 0; ##P<0.01 vs. Pressure +/Nicotine 0; ###P<0.001 vs. Pressure +/Nicotine 0; ####P<0.0001 vs. Pressure +/Nicotine 0.

### DETAILED DESCRIPTION

The present invention in various aspect and embodiments provides compositions and methods for preventing or treating pancreatitis. The invention in various embodiments comprises administering a composition comprising an effective amount of phosphate to the subject so as to treat or prevent pancreatitis in the subject. In the various aspects and embodiments, the method may comprise, consist of, or consist essentially administration of phosphate (or salt thereof) for prevention or treatment of pancreatitis.

Pancreatitis is a debilitating inflammatory disease of the pancreas that causes substantial morbidity and mortality. Alcohol consumption accounts for about 40% of human acute pancreatitis. Another approximately 40% of cases are due to gallstone obstruction of the pancreatic duct (referred to herein as “gallstone pancreatitis” or “biliary pancreatitis”). The remaining causes of acute pancreatitis include drugs and toxins, metabolic abnormalities such as hypercalcemia and hyperlipidemia, trauma, iatrogenic maneuvers including endoscopic retrograde cholangiopancreatography (ERCP), and genetic mutations (e.g., hereditary pancreatitis, cystic fibrosis). Patients at most predictable risk for acute pancreatitis are those undergoing endoscopic procedures.

Chronic or recurrent injury to the pancreas including drinking alcohol can lead to chronic pancreatitis. Other causes include hereditary pancreatitis, tropical pancreatitis, and other less common or unknown causes. Patients with chronic pancreatitis may suffer from persistent symptoms or experience recurrent episodes (i.e., attacks). However, the frequency may be predictable and lend itself to preventative strategies.

In a retrospective study at Duke University Medical Center, it was observed that low serum phosphate levels correlate with pancreatitis severity. Normal serum phosphate levels are 2.5 to 4.5 mg/dL in adults, while mild hypophosphatemia (which is not conventionally treated), has serum phosphate in the range of 2.0 to 2.4 mg/dL. However, changes in serum phosphate are dynamic. For example, pancreatitis patients may present with normal or even hyperphosphatemia that is quickly followed by hypophosphatemia, which may remain low as the condition progresses. Therefore, phosphate treatment early in the course (or even prophylactically before serum phosphate levels decline, and before hypophosphatemia develops), can be used for treating pancreatitis patients in accordance with the disclosure. Specifically, accordance with this disclosure, it is believed that phosphate replacement restores ATP levels in the pancreas and limits pancreatic damage.

In some embodiments, the subject is at risk for developing pancreatitis. In these embodiments, the patient may or may not have clinical hypophosphatemia, but phosphate supplementation will protect from organ injury. In these embodiments, the subject may be at risk of pancreatitis of various etiologies, including biliary pancreatitis or gallstone pancreatitis. In other embodiments, the subject is an alcohol user and at risk of alcoholic pancreatitis. In still other embodiments, the subject has hypertriglyceridemia or hypercalcemia, which are risk factors for pancreatitis. In still other embodiments, the subject is undergoing treatment with one or more active agents that increase risk of pancreatitis such as: glucagon-like peptide 1 receptor agonists (e.g., albiglutide, dulaglutide, exenatide, extended-release exenatide, liraglutide, lixisenatide, semaglutide), dipeptidyl peptidase—4 inhibitors (DPP-IV inhibitors) (e.g., sitagliptin, saxagliptin, linagliptin, and alogliptin), asparaginase, HIV-1 drugs, hydrochlorothiazide, azathioprine, etc. In still other embodiments, the subject has a biological or genetic predisposition to develop pancreatitis, such as cystic fibrosis (“hereditary pancreatitis”). In still other embodiments, the subject will undergo a post-endoscopic retrograde cholangiopancreatography (ERCP) procedure, which often induces pancreatitis. In still other embodiments, the subject has an autoimmune disease that predisposes to pancreatitis.

When treating a subject at risk for pancreatitis, the subject may or may not have clinical hypophosphatemia. As used herein, clinical hypophosphatemia is defined as a serum phosphate level of less than 2.0 mg/dL for adults. In some embodiments, the subject will have a normal serum phosphate level (defined herein as the range 2.5 to 4.5 mg/dL for adults) or mild (subclinical) hypophosphatemia (defined herein as a serum phosphate level of 2.0 to 2.4 mg/dL for adults). In accordance with embodiments of the invention, phosphate treatment can be provided prophylactically before serum phosphate levels decline and before hypophosphatemia (subclinical or clinical) develops.

In some aspects, the subject presents with pancreatitis, including pancreatitis of any etiology, including gallstone pancreatitis, biliary pancreatitis, alcoholic pancreatitis, acute pancreatitis associated with drugs or toxins, metabolic abnormalities such as hypercalcemia and hyperlipidemia, trauma (including surgery), autoimmune disease, iatrogenic maneuvers including endoscopic retrograde cholangiopancreatography (ERCP), and genetic mutations (e.g., hereditary pancreatitis, cystic fibrosis). In some embodiments the pancreatitis is idiopathic pancreatitis.

The subject may have chronic or acute pancreatitis. Patients with chronic pancreatitis may suffer from persistent symptoms or experience recurrent episodes (i.e., attacks). Acute pancreatitis may be mild acute pancreatitis, moderately severe pancreatitis, or severe pancreatitis, as determined by the Atlanta Revision scoring system. See, Chatila AT, et al., Evaluation and management of acute pancreatitis, World J. Clin. Cases May 6; 7(9): 1006-1020. For example, mild acute pancreatitis is defined as the absence of organ failure, and absence of local complications. Moderately severe pancreatitis is defined as the presence of local complications and/or transient organ failure for less than 48 hours. Severe pancreatitis is defined as persistent organ failure for greater than 48 hours. In accordance with embodiments of the invention, administration of phosphate early (e.g., by i.v.) to patients presenting with mild acute pancreatitis, can prevent or slow progression of the disease. As described above, a subject presenting with mild acute pancreatitis may or may not have clinical hypophosphatemia, and phosphate treatment may be given before serum phosphate levels decline and before hypophosphatemia (subclinical or clinical) develops. In various embodiments, the subject does not have hyperphosphatemia at the time phosphate is administered.

In some embodiments, before undergoing phosphate therapy, the subject is tested for phosphate levels, and phosphate is administered if the patient does not have hyperphosphatemia (defined herein as a serum phosphate level of greater than 4.5 mg/dL for adults). Generally, it would not be prudent to treat a patient with hyperphosphatemia with supplemental phosphate. Further, in some embodiments, the subject does not have renal insufficiency (acute kidney injury, or AKI), since phosphate treatment is not recommended in patients with evidence of renal insufficiency (e.g., elevated serum creatinine).

In some embodiments, the treatment with phosphate comprises sodium phosphate and/or potassium phosphate. For example, in some embodiments, the treatment involves intravenous administration with, for example, sodium phosphate. For example, the sodium phosphate injection can be provided as a concentrated solution containing a mixture of monobasic sodium phosphate and dibasic sodium phosphate in water for injection. Sodium phosphate compositions for intravenous injection are known in the art. In some embodiments, the sodium phosphate injection is provided intermittently as needed to keep phosphate levels in the normal range or when pancreatitis symptoms present. In some embodiments, i.v. phosphate treatment is administered to the subject prior to or after undergoing ERCP to protect the subject from developing pancreatitis.

In some embodiments, two to four liters of fluid are generally administered over the first 24 hours to patients admitted to the hospital with acute pancreatitis. This rehydration may employ a phosphate-containing pancreatitis rehydration solution (e.g., for i.v.) that comprises a mixture of NaCl and sodium and/or potassium phosphate. For example, in some embodiments the solution comprises from about 50 to about 165 mmol/L NaCl and about 5 to about 300 mmol/L sodium and/or potassium phosphate with a physiological pH (e.g., about 7.3 to about 7.5). In one embodiment, the solution comprises from about 100 to about 150 mmol/L NaCl and about 15 to about 75 mmol/L sodium and/or potassium phosphate, with a physiological pH of about 7.4). In other embodiments, the solution comprises from about 120 to about 140 mmol/L NaCl and about 25 to about 55 mmol/L sodium and/or potassium phosphate, with a physiological pH of about 7.4). In some embodiments, the rate of administration is about 10 mL/kg/h over about 8 hours, which is optionally followed by about 3 mL/kg/h for next about 16 hours. Of course, parameters for administration can be determined by the attending physician.

In other embodiments, phosphate supplementation is administered orally. For patients at risk of pancreatitis, or having chronic pancreatitis or prior episode of acute pancreatitis, oral phosphate supplementation protects from pancreatitis or further episodes or severity thereof. Phosphates are used as dietary supplements for patients who are unable to get enough phosphorus in their regular diet, usually because of certain illnesses or diseases. In various embodiments, the oral supplementation is provided in tablet or capsule form, or may be provided in liquid form, and can be administered in a daily dose of from about 300 mg to about 1500 mg, or in some embodiments, in the range of about 500 mg to about 1250 mg, or in some embodiments, in the range of about 500 mg to about 1000 mg. Alternatively, the subject can be administered the supplement at least every other day, at least every third day, or at least once per week as needed to maintain normal phosphate levels. In some embodiments, the phosphate supplement is administered for at least about two weeks, or at least about four weeks (or one month), or at least about two months, or at least about six months. In some embodiments, phosphate supplementation is provided continually as a dietary supplement.

In some embodiments, the patient is further administered one or more agents that may act additively or synergistically to protect from pancreatitis. Such agents are selected from one or more of: an alpha 7 nicotinic acetylcholine receptor agonist, TRPV4 antagonist, an anticoagulant, pancreatic enzyme inhibitor, magnesium, non-steroidal anti-inflammatory drug (NSAID), calcineurin inhibitor, and calcium channel blocker. Such agents may be administered in the same composition as the phosphate (together with one or more pharmaceutically acceptable carriers or excipients) or separate composition. In various embodiments, such agents are administered by i.v. or orally, or by other appropriate route including but not limited to the transdermal. For example, one or more agents may be administered using a transdermal patch.

Exemplary alpha 7 nicotinic acetylcholine receptor agonist is nicotine or a nicotinic agonist, such as GTS-21. Nicotinic agonists in some embodiments are administered in the same or separate composition as the phosphate. Nicotinic agonists may be administered by i.v., or by transdermal patch in some embodiments. GTS-21 (DMXBA) is a derivative of the natural product anabaseine that acts as a partial agonist at neural nicotinic acetylcholine receptors. GTS-21 is 3-2,4-dimethoxybenzylidene anabaseine. As demonstrated herein, GTS-1 and nicotine can protect from pancreatitis. GTS-1 can be administered by any suitable route, including in a co-formulation with phosphate or as a separate pharmaceutical composition.

Exemplary TRPV4 antagonists include GSK2798745, GSK101, GSK205, and analogs thereof. See Kanju et al., Small molecule dual-inhibitors of TRPV4 and TRPA1 for attenuation of inflammation and pain, Scientfic Reports 6, Article 26894 (2016). TRPV4 agonists may be administered in the same or separate composition from the phosphate.

Exemplary anticoagulants include heparin, warfarin, as well as novel oral anticoagulants (NOACs) [(aka direct oral anticoagulants (DOACs)] including thrombin inhibitors (e.g., dabigatran) and factor Xa inhibitors (apixaban, rivaroxaban, and edoxaban). Anticoagulants may be administered in the same or different composition as the phosphate.

In some embodiments, pancreatic enzyme inhibitors are administered, particularly inhibitors of trypsin, chymotrypsin, lipase, and/or elastase. Various such inhibitors are known in the art.

Exemplary non-steroidal anti-inflammatory drugs (NSAIDs) include aspirin, naproxen, ibuprofen, as well as indomethacin (e.g., intra-rectal indomethacin). NSAIDs may be administered in the same or separate composition as the phosphate.

Exemplary calcium channel blockers include pharmacological inhibitors of the Orai1 channel (also known as the calcium release activated channel (CRAC) (Wen et al. Gastroenterology. 2015 August; 149(2): 481-492.e7.

In still other aspect, the invention provides a pharmaceutical composition comprising an effective amount of a phosphate salt (such as a sodium and/or potassium phosphate salt) and one or more of: an α7 nicotinic acetylcholine receptor agonist, TRPV4 antagonist, an anticoagulant, pancreatic enzyme inhibitor, magnesium, non-steroidal anti-inflammatory drug (NSAID), calcineurin inhibitor, and calcium channel blocker. Exemplary agents are further described above, which can be used in this aspect. Such compositions can be formulated for any administration route, including intravenous administration, intradermal administration, and oral administration. Other potential routes include aerosol, intranasal, buccal, injection (such as within the intrapancreatic duct and/or celiac artery) parenteral, subcutaneous, intramuscular, intraperitoneal and rectal. These compositions can provide novel formulations for treating or preventing pancreatitis, and may have additive or synergistic properties for treating or preventing pancreatitis, as compared to the agents individually.

As used herein, a “pharmaceutically acceptable excipient” or “pharmaceutically acceptable carrier” are used interchangeably, and include in the case of intravenous administration, sterile distilled water, saline, and buffered solutions, many of which are known in the art.

In the case of oral supplementation, formulations of agents and supplements may be prepared with physiologically acceptable carriers, excipients, as is well known in the art (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

An effective amount for a particular subject/patient may vary depending on factors such as the severity of the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

Methods for co-administration with additional therapeutic agent(s) are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

### Definitions

Unless otherwise defined, all technical terms used herein have the same meaning as commonly understood by one of ordinary skill in the art to which this disclosure belongs.

Articles “a” and “an” are used herein to refer to one or to more than one (i.e. at least one) of the grammatical object of the article. By way of example, “an element” means at least one element and can include more than one element.

“About” as used herein means ±10% of an associated numerical value.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein, “treatment” or “treating” refers to the clinical intervention made in response to a disease, disorder or physiological condition manifested by a patient or to which a patient may be susceptible. The aim of treatment includes the alleviation or of symptoms, slowing or stopping the progression or worsening of a disease, disorder, or condition and/or the remission of the disease, disorder or condition. The term “prevention” refers to the prevention of symptoms of a disease, disorder, or condition from manifesting in a subject.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals (e.g., mammals). Preferably, the subject is a human patient that is suffering from, or at risk of, pancreatitis.

The following examples are provided by way of illustration and not by way of limitation.

### Examples

### Example 1: Hypophosphatemia is Associated with Pancreatitis

Acute pancreatitis affects more than 200,000 people in the United States and is common among military veterans. A hallmark of acute pancreatitis is systemic injury and multi-organ failure leading to mortality in 3-20% of the patients. There are currently no treatments for acute pancreatitis. As demonstrated herein, acinar cell injury is associated with mitochondrial dysfunction and ATP depletion and supplementing phosphate has a protective and ameliorating effect in both in vitro and in vivo models of pancreatitis.

The pancreas is a metabolically active organ due to abundant synthesis of digestive enzymes, which requires mitochondrial production of ATP as a source of energy. Disruption of these processes through pancreatic injury can cause pancreatitis and contribute to pancreatitis severity. Phosphate is required for mitochondrial oxidative phosphorylation and clinical hypophosphatemia is associated with decreased ATP production and mitochondrial dysfunction. Thus, phosphate availability is believed to be necessary for pancreatic acinar cell function. Indeed, approximately 70% of patients with acute pancreatitis experience hypophosphatemia.

As described herein, mitochondrial dysfunction accompanies pancreatic injury and hypophosphatemia is associated with increasing severity of pancreatitis. Moreover, this disclosure demonstrates that phosphate supplementation reduces pancreatitis severity, and therefore, acute pancreatitis can be ameliorated by phosphate treatment.

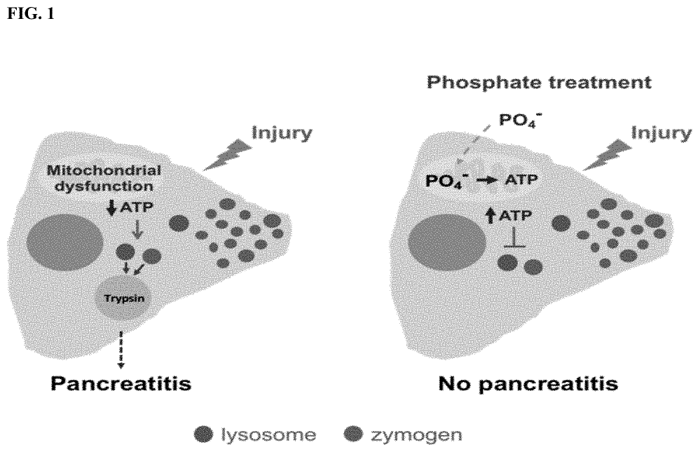

FIG. 1 illustrates the concept of rescue from pancreatic injury by phosphate supplementation, which may stabilize mitochondrial function and ATP production in injured acinar cells. Pancreatic injury causes mitochondrial dysfunction and reduced ATP production. Loss of ATP leads to intracellular zymogen activation, and pancreatitis. In accordance with this disclosure, it is proposed that phosphate depletion contributes to mitochondrial dysfunction and that phosphate treatment will preserve mitochondrial function and ameliorate pancreatitis.

In a retrospective analysis of 109 patients admitted to Duke University Hospital from Sep. 1, 2016 to Aug. 31, 2018, it was observed that hypophosphatemia was not only common in acute pancreatitis but was associated with worsening pancreatitis severity. Since phosphate is required for mitochondrial oxidative phosphorylation and clinical hypophosphatemia is associated with decreased ATP production and mitochondrial dysfunction, we propose that phosphate availability, which is necessary for pancreatic acinar cell function, may underlie susceptibility to pancreatitis.

In the retrospective study, inclusion criteria included diagnosis of acute pancreatitis, and at least 18 years of age; exclusion criteria included: age less than 18 years; chronic or recurrent pancreatitis; end stage renal disease; hyperparathyroidism; diabetic ketoacidosis, and sepsis. Hospital admission dates were between Sep. 1, 2016 and Aug. 31, 2018. Hypophosphatemia was defined in two ways: 1) a “first” in-hospital phosphate level<2.5 mg/dL with no drop in phosphate level>1.0 mg/dL; 2) a maximum drop of >1.0 mg/dL in phosphate level between the “first” in-hospital level and the minimum phosphate level obtained during the hospital stay. Patients who did not meet either of these criteria were considered to have normal phosphate levels.

In a preliminary data analysis of the 109 patients, 31% of patients studied met criterion 1 above for hypophosphatemia and 17% of patients met criterion 2. It was hypothesized that low serum phosphate levels may adversely affect the pancreas by limiting phosphate (Pi) availability required for ATP synthesis. In order to assess the relationship between serum phosphate levels and clinical outcome, the association between hypophosphatemia status and the presence of severe pancreatitis was tested. Differences were observed in the proportions of patients with severe pancreatitis by hypophosphatemia status; 10.7% of patients without hypophosphatemia were determined to have severe pancreatitis versus 23.5% and 52.6% for each of the hypophosphatemia groups, respectively (X² p<0.001). These findings suggest that there is an association between clinical hypophosphatemia and pancreatitis severity but it is not possible to determine if low phosphate levels predispose to pancreatitis or if pancreatitis causes hypophosphatemia. To address this issue, animal studies in to evaluate the effects of phosphate on the development, severity and resolution of acute pancreatitis were designed.

### Example 2: Caerulein-Induced Pancreatitis Model

Caerulein-induced pancreatitis in mice is an established model of acute experimental pancreatitis. Hyperstimulation of the pancreas by cholecystokinin or its analogue caerulein is a commonly used model of experimental acute pancreatitis in rodents. Caerulein produces mild to moderate, non-fatal pancreatitis that resolves spontaneously.

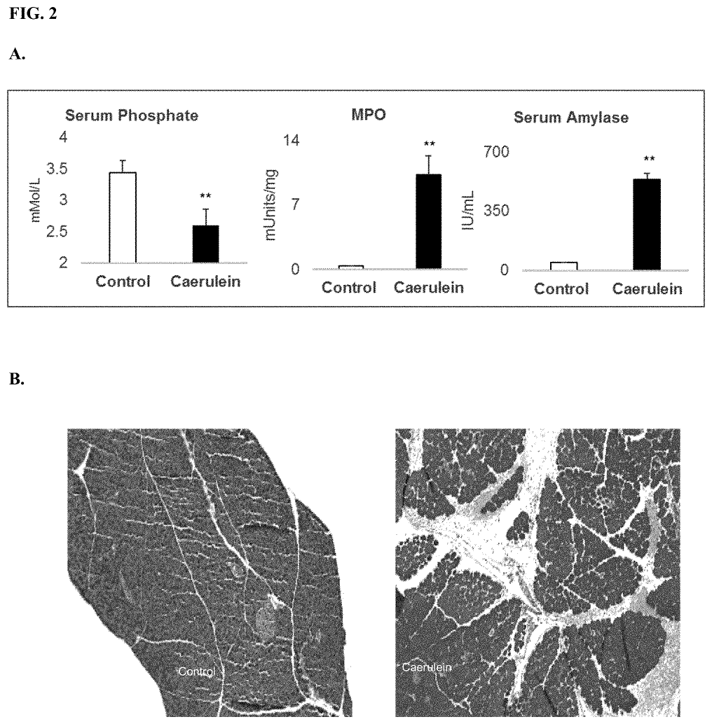

In this mouse model, it was demonstrated that pancreatitis is associated with hypophosphatemia (FIG. 2A, B). Wild type mice were treated with 6 hourly injections of caerulein (50 ng/g). Serum phosphate levels were measured 7 hours after the initial caerulein injection. Pancreatic myeloperoxidase (MPO) reflects neutrophil infiltration and is a quantitative measure of pancreatic inflammation (FIG. 2A). Serum amylase and pancreatic MPO levels (FIG. 2A) and histology (FIG. 2B) were evaluated 7 hours after the initial caerulein injection. **P<0.01 vs. control. Control group n=4 and caerulein group n=5.

Realizing the high metabolic demands of the pancreas and the critical requirement for proper digestive enzyme synthesis and packaging to maintain normal intracellular homeostasis, it was proposed that supplementing phosphate could improve acinar cell function and perhaps limit the development of pancreatitis. Moreover, hypophosphatemia develops during the course of pancreatitis thereby reducing the availability of phosphate which is necessary for normal cellular function. It was proposed that improving phosphate availability would reduce pancreatic injury. To test this hypothesis, sodium phosphate was adminstered to caerulein-treated mice.

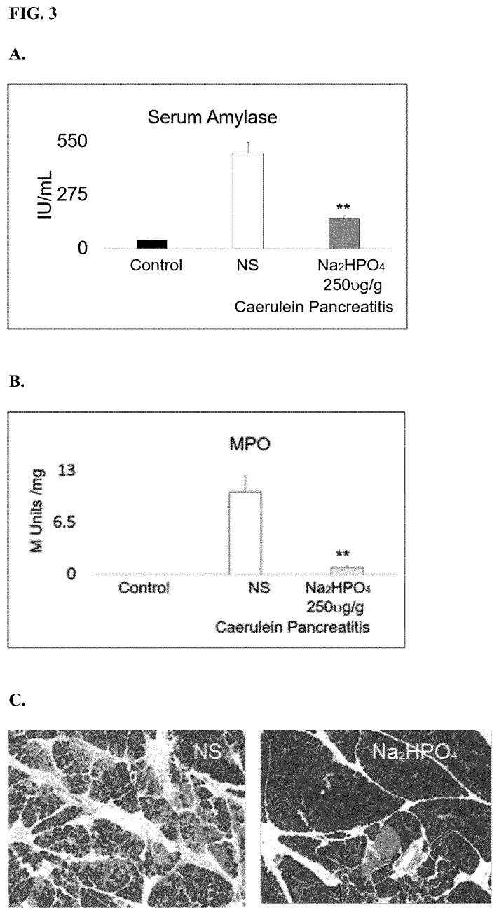

As shown in FIGS. 3A-3C, phosphate supplementation reduced all parameters of pancreatitis following caerulein administration including serum amylase levels (FIG. 3A), pancreatic MPO levels (FIG. 3B) and pancreatic histology (FIG. 3C). Specifically, mice (C56BL/6 adult mice) were treated with 6 hourly injections of caerulein sulfate (50 ng/g) by intraperitoneal injection. Sodium phosphate (250 μg/g Na₂HPO₄) and normal saline (NS) were given 1 hour after the first injection. The study was terminated 1 hour after the last caerulein injection at which time, blood and pancreas tissue are collected for measurements of serum amylase, and pancreatic edema, MPO, and histological pancreatitis severity. Pancreatic myeloperoxidase (MPO) and serum amylase levels were measured by commercially available assays. **P<0.01 vs. normal saline in caerulein-treated mice.

To evaluate the effects of phosphate on acinar cell function pancreatic acini in vitro were examined High concentrations of caerulein in vitro induce pancreatic injury similar to the effects observed in vivo and this method has been used to study direct effects on pancreatic acinar cells. Specifically, caerulein causes pancreatitis by hyperstimulating the acinar cells through the CCK1 receptor. In isolated pancreatic acini, caerulein causes trypsin activation, generation of the proinflammatory cytokine cytosolic interleukin-10, and cell necrosis as manifest by LDH release. Each of these pathological results is seen with caerulein hyperstimulation in vivo.

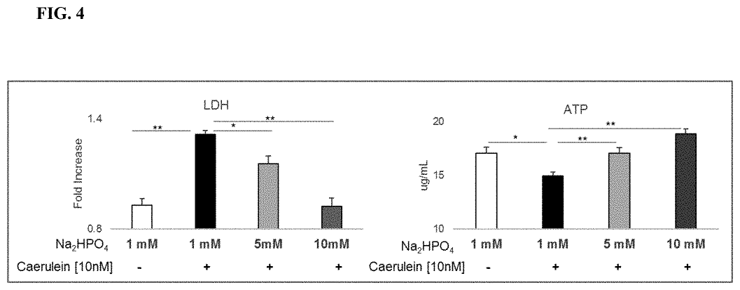

It was observed that phosphate supplementation, independent of its buffering effects, reduced caerulein-induced LDH release, a marker of cell damage (FIG. 4). In addition, it was sought to determine if lack of ATP contributes to pancreatitis and whether the protective effects of phosphate are through restoration of intracellular ATP levels. Initial studies by the inventors have indicated that caerulein decreases ATP production, and phosphate supplementation in a dose-dependent manner restores cytosolic ATP (data not shown). FIG. 4 shows the effects of phosphate on caerulein-induced injury (left panel) and reduction of cytosolic ATP levels (right panel) in isolated pancreatic acini. Isolated mouse pancreatic acini were treated with caerulein in buffer containing Na₂HPO₄ concentrations of 1-10 mM. Supernatant LDH was measured by commercial LDH assay. Cytosolic ATP levels were measured using a commercially available fluorometric assay. *P<0.05, **<0.01, ***<0.001. Sodium phosphate reduced the effects of caerulein-induced injury and restored ATP levels in a dose-dependent fashion.

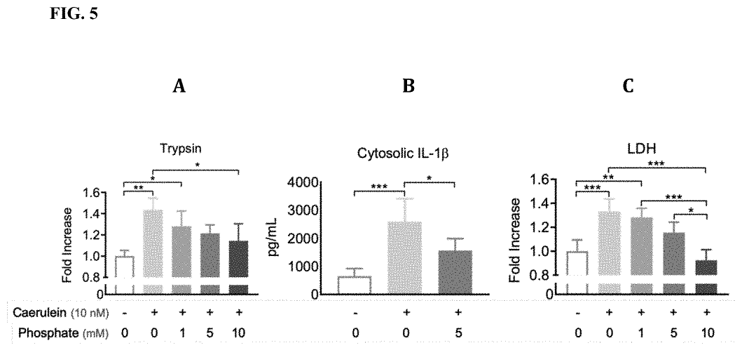

Further, as shown in FIG. 5, phosphate protects against caerulein-induced damage in isolated pancreatic acini. Pancreatic acini were incubated in phosphate-free media or media containing 1, 5, or 10 mM sodium phosphate (NaH₂PO₄). All solutions were adjusted to pH 7.4. A supramaximal concentration of caerulein (10 nM) was added for 30 minutes as indicated after which media were analyzed for release of trypsin, IL-1β and LDH. (*P<0.05, **P<0.01, ***P<0.001).

Thus, phosphate deficiency can lead to reduced ATP production and organ dysfunction. Pancreatic acinar cells are highly metabolically active and require a high level of ATP to maintain proper cellular function including zymogen granule and lysomal integrity, enzyme secretion and autophagy. Under conditions of phosphate deficiency and reduced ATP synthesis, all of these processes are subject to fail leading to pancreatitis.

Moreover, the potential protective effects of phosphate in vitro under conditions that resemble the same processes that produce pancreatitis in vivo were evaluated. It was found that, in a dose-dependent manner, phosphate improved each of the measures of acinar cell damage.

Next, an investigation as to whether mitochondrial dysfunction in pancreatitis can be ameliorated by supplemental Pi was conducted. Proper mitochondrial function relies heavily upon proper regulation of cytosolic calcium concentration ([Ca²⁺]ᵢ). Disruption of [Ca²⁺]ᵢ homeostasis that produces high levels leads to loss of the mitochondrial membrane potential which is essential for ATP production. Inorganic phosphate (Pi) is also required for ATP synthesis.

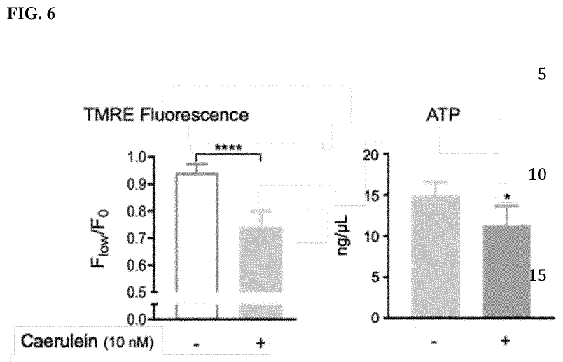

Mitochondrial membrane potential was measured in isolated pancreatic acini using tetramethylrhodamine ethyl ester (TMRE) fluorescence. As shown in FIG. 6, caerulein hyperstimulation caused mitochondrial depolarization and reduced ATP production.

Specifically, isolated pancreatic acini were loaded with the mitochondrial fluorescent dye TMRE and incubated in phosphate-free buffer with a supramaximal concentration of caerulein (10 nM). TMRE fluorescence and intracellular ATP levels were measure after 30 minutes. *P<0.05, ****P<0.0001.

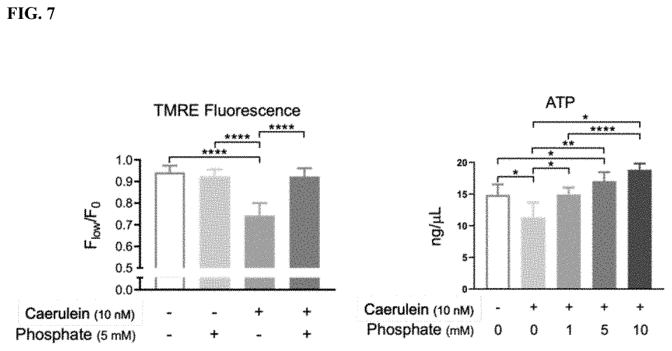

These functions were phosphate dependent, as NaH₂PO₄ supplementation dose-dependently restored both mitochondrial function and ATP synthesis (FIG. 7). Isolated pancreatic acini loaded with TMRE were incubated in buffer with 0 or 5 mM phosphate (left panel). Effects of increasing phosphate concentrations on ATP production are shown in the right panel. *P<0.05, **P<0.01, ****P<0.0001. These findings illustrate the requirement for Pi in maintaining and restoring mitochondrial function and ATP synthesis under the stress of caerulein hyperstimulation.

### Example 3: Pressure-Induced Pancreatitis Models and Assays

To demonstrate a potential role of phosphate supplementation in protecting from pancreatic injury, mice were fed a low phosphate diet, and their increased susceptibility to pancreatic injury was evaluated. Endoscopic retrograde pancreatography (ERCP) is a common cause of human pancreatitis and results from injection of radiocontrast material into the pancreatic duct at high pressure. For this study, a mouse model of this form of pancreatitis was employed.

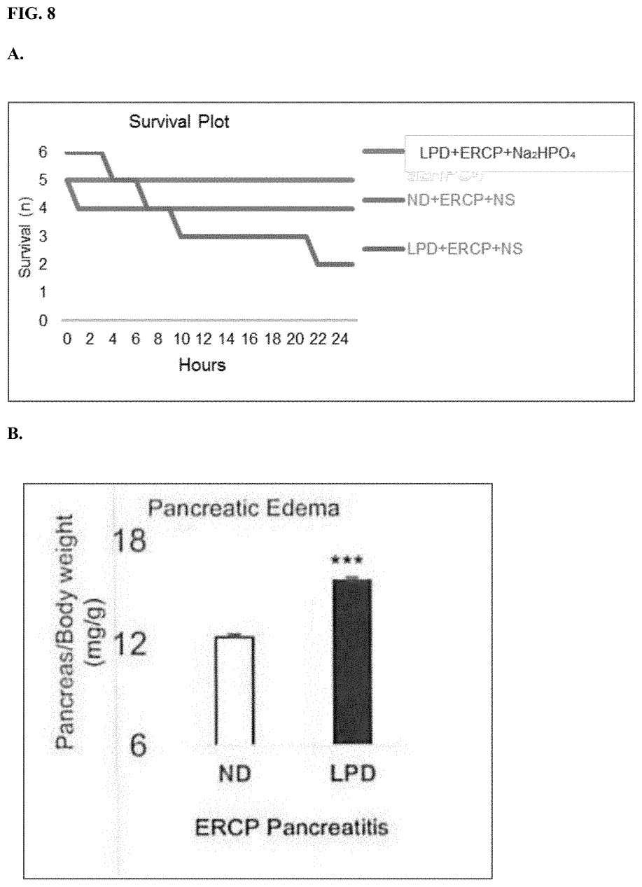

Normal serum phosphate levels in the mouse are 3.2-4 mmol/L. Mice were maintained on the control or low phosphate diets as pancreatitis was induced. Animals on a low phosphate diet had low serum phosphate levels of 2 mmol/L and were more susceptible to pancreatitis with greater mortality, as compared to animal on a normal diet (FIG. 8A, B). Specifically, to induce hypophosphatemia, mice were placed on a low phosphate diet (LPD) for 3 weeks. ERCP pancreatitis was then induced in control mice on a normal diet (ND) and hypophosphatemic mice (C57BL/6) on a low phosphate diet (LPD), via retrograde pancreatic duct infusion. Sodium phosphate (200 μg/g) or normal saline (NS) was given by intraperitoneal injection 1 and 4 hours after induction of ERCP pancreatitis. Mice were scarified 24 hours after the surgery and pancreatitis parameters measured. ***P<0.001 vs. normal diet. As shown in FIGS. 8A and 8B, LPD exacerbated pancreatic injury, while sodium phosphate supplementation protected the mice from this injury (FIG. 8A, B).

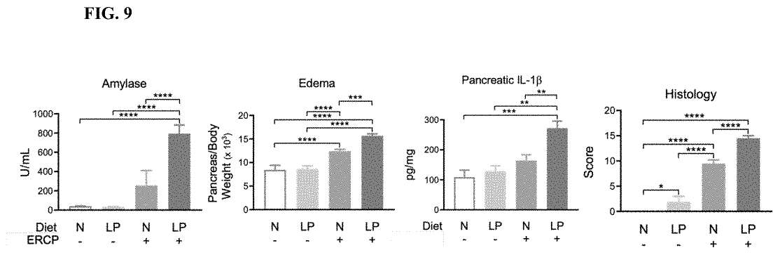

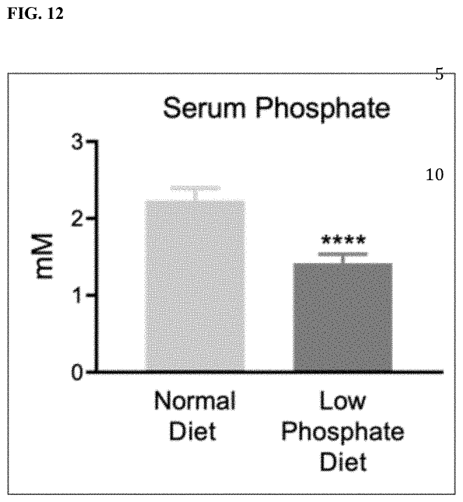

Further, in a similar experiment, as shown in FIG. 9, pancreatic duct injection induced pancreatic inflammation in normal chow fed mice as manifest by increased serum amylase, pancreatic edema, IL-10, and pancreatic histology scores. Each of these parameters was significantly worse in mice fed the low phosphate diet. These data suggest that hypophosphatemia leads to more severe pancreatitis. *P<0.05, **P<0.01, ***P<0.001, ****P<0.0001. Normal serum phosphate levels averaged 2.2 mM, and after three weeks on the low phosphate diet serum levels of phosphate declined to 1.4 mM. See FIG. 12.

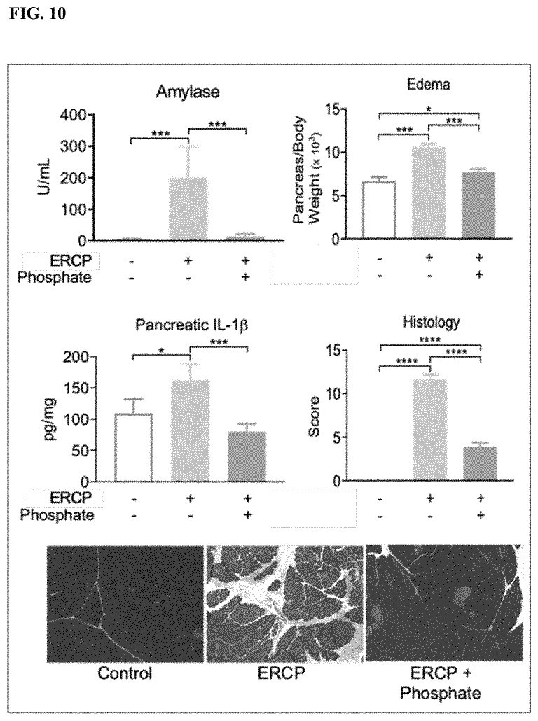

Further, it was demonstrated that phosphate can ameliorate pancreatitis after the injury is initiated (FIG. 10). As shown, duct injection produced acute pancreatitis. To determine if phosphate protected against ERCP-induced pancreatitis, we treated animals with sodium phosphate (NaH₂PO₄ 50 mg/kg) administered by IP injection 2 and 4 hours after surgery. As shown, phosphate administration significantly ameliorated these parameters of acute pancreatitis. It is important to note that phosphate was administered after pancreatic duct injection, raising the possibility that phosphate could be used as a treatment for pancreatitis once the disease process has been initiated. FIG. 10 shows pancreatitis parameters and pancreatic histology measured 24 hrs after induction of pancreatitis. *P<0.05, ***P<0.001, ****P<0.001 (n=3-8).

The pancreas is highly sensitive to pressure and elevated pancreatic duct pressure is a major cause of acute pancreatitis. Intraductal pancreatic pressure is increased during the clinical procedure endoscopic retrograde cholangiopancreatography (ERCP) when radiocontrast dye is injected into the pancreatic duct to visualize the pancreas. Acute pancreatitis develops in up to 20% of high risk patients undergoing this procedure. It was observed that the pancreatic acinar cell senses pressure through the mechanically activated ion channel Piezol, and activation of Piezol on acinar cells by pressure or a Piezol-specific agonist, induced acute pancreatitis. Moreover, mice with acinar cell-specific deletion of Piezol were protected from pressure-induced pancreatitis (data not shown).

Pathological activation of Piezol leads to [Ca²⁺]ᵢ overload, mitochondrial dysfunction, trypsinogen activation, and acinar cell necrosis. Moreover, Piezol activation explains how elevation of intrapancreatic duct pressure (e.g., ERCP or gallstone impaction) causes pancreatitis. Piezol can be activated by mechanical pushing or shear stress. A method for studying the effects of shear stress on isolated pancreatic acini in vitro was developed. Pancreatic acini are cultured on Matrigel and placed into a fluid flow chamber. Cells can be loaded with either the calcium sensitive dye, Calcium 6-QF (Molecular Devices), or the active trypsin enzyme substrate BZiPAR [rhodamine 110, bis(CBZ-L-isoleucyl-L-prolyl-L-arginine amide)] (10 μM) and imaged using a fluorescence microscope while fluid flow is regulated to control the level of shear stress.

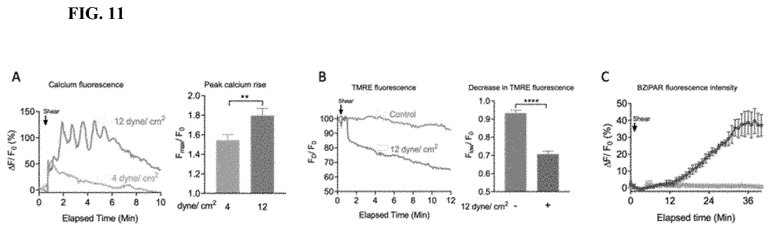

FIG. 11 shows that shear pressure causes sustained elevation in [Ca²⁺], mitochondrial depolarization, and trypsin activation in pancreatic acinar cells. In FIG. 11(A), isolated pancreatic acini were loaded with Calcium 6-QF, placed in a fluid flow chamber and imaged by fluorescence microscopy. Shear flow of culture media was applied at a force of 4 or 12 dyne/cm². A representative image shows a typical intracellular calcium response. The bar graph shows the peak calcium fluorescence from 30 cells (**P<0.01). In FIG. 11(B), TMRE fluorescence was used to assess mitochondrial function. A decrease in TMRE fluorescence represents mitochondrial membrane depolarization and loss of function. The effects of shear flow pressure at 12 dyne/cm² (lower line) is compared to no flow control (upper line). The bar graph shows the average decrease in TMRE fluorescence of 20 cells (****P<0.0001). FIG. 11(C) Intracellular trypsin activation was demonstrated by an increase in BZiPAR fluorescence following shear flow at 12 dyne/cm² compared to control acini not subjected to flow.

As shown in FIG. 11(A)-(C), subjecting acinar cells to fluid shear stress at 12 dyne/cm² produced a prolonged increase in [Ca²⁺]ᵢ similar to that seen with supramaximal, pathological concentrations of caerulein and was sufficient to cause trypsin activation. Shear pressure also led to mitochondrial membrane depolarization indicating mitochondrial dysfunction. These effects are specific for Piezol since changes were not observed in Piezol1^{aci} knockout mice. Yoda1 is a selective agonist for Piezol and can be used to simulate pressure by chemically activating Piezol. Yoda1 (25 μM) caused an elevation in [Ca²⁺]ᵢ like that of shear stress and increased LDH release simulating a deleterious effect of pressure on acinar cells. In order to determine if phosphate might protect against pressure-induced pancreatitis, we observed that phosphate supplementation reduced Yoda1-stimulated elevations in [Ca²⁺]ᵢ and LDH release.

### Example 3: Phosphate Treatment Ameliorates Alcohol-Induced Pancreatitis

Alcohol consumption accounts for 40% of human acute pancreatitis and despite intensive investigation the pathogenesis of alcohol-induced pancreatitis remains poorly understood. There are no effective treatments for human pancreatitis caused by ethanol consumption. Up to 30% of alcoholic patients admitted to the hospital have hypophosphatemia (serum phosphate<2.4 mg/dL). The hypophosphatemia is commonly caused by malnutrition, however, alcohol also impairs phosphate absorption in the intestine. Following hospitalization, refeeding may exacerbate hypophosphatemia and alcohol withdrawal may create respiratory alkalosis which can exacerbate hypophosphatemia. Therefore, several factors render alcoholic patients susceptible to hypophosphatemia.

Studies designed to unveil the mechanisms underlying alcohol-induced pancreatitis have generally focused on (i) direct toxic effects of ethanol or (ii) immune responses to ethanol ingestion. A major limitation to scientific progress in understanding alcohol-induced pancreatitis has been the lack of suitable models for studying the disease. In normal laboratory animals, alcohol feeding does not cause pancreatitis. To overcome this problem, alcohol has been combined with other experimental methods for inducing pancreatitis to determine whether alcohol worsens pancreatitis severity. Alternatively, alcohol has been administered parenterally with metabolites of non-oxidative ethanol metabolism (e.g., fatty acid ethyl esters) to induce pancreatitis. Unfortunately, none of these models completely recapitulates alcoholic pancreatitis.

While administration of alcohol alone does not produce pancreatitis in normal mice, as shown herein, alcohol rapidly induces pancreatitis when mice are fed a low phosphate diet, consistent with the concept that hypophosphatemia sensitizes the pancreas to alcohol. Importantly, phosphate supplementation also prevented alcohol-induced pancreatic injury. These findings confirm that phosphate availability is critical for acinar cell health and phosphate supplementation can improve pancreatitis severity.

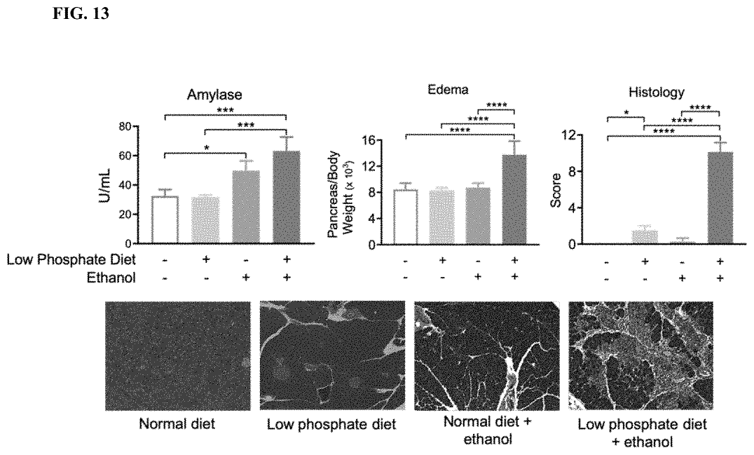

These findings raise the question of whether phosphate deficiency contributes to the progression of pancreatitis. This may be particularly relevant to alcohol-induced pancreatitis, given that alcoholic patients are susceptible to hypophosphatemia due to nutritional deficiencies and the effects of alcohol ingestion on phosphate absorption. Therefore, it was hypothesized that low phosphate levels may be predispose to alcohol-induced pancreatic injury. To test this hypothesis, we placed mice on a low phosphate diet and determined their susceptibility to ethanol. Animals were placed on a chow diet containing low amounts of phosphate (Pi) for three weeks. The low phosphate diet (Envigo #TD.140659) was comprised of 0.02% Pi compared to normal chow diet which contained 1% Pi. Serum levels of phosphate declined in mice on a low phosphate diet from normal values of 2.2 mM to 1.4 mM (FIG. 12). In order to determine if the diet rendered mice more sensitive to ethanol, mice were administered ethanol by orogastric gavage at 3 mg/kg (30% ethanol at 10 mL/kg) daily for five days (Control mice were gavaged with water). Mice were euthanized 12 hours after the last dose of ethanol. Serum amylase levels, pancreatic edema, and histological scoring for pancreatitis severity are shown in FIG. 13. The pancreatitis severity scoring system is a composite of edema, necrosis, inflammatory cell infiltration and hemorrhage severity as previously published. As shown in FIG. 13, neither the low phosphate diet nor ethanol alone caused pancreatitis. However, administration of ethanol to animals on a low phosphate diet induced marked evidence of acute pancreatitis. Thus, low phosphate diet renders mice susceptible to ethanol pancreatitis.

The observation that the low phosphate diet rendered mice susceptible to ethanol-induced pancreatitis was considered to be a remarkable finding. Historically, it has been very difficult to induce acute pancreatitis in rodents by administering ethanol. Therefore, an approach combining a low phosphate diet with ethanol gavage may provide a useful model for studying the pathogenesis of alcohol-induced injury. In addition, these findings point to a key role for phosphate in the evolution of pancreatitis and raise the possibility that restoring serum phosphate to normal levels might protect against acute pancreatitis.

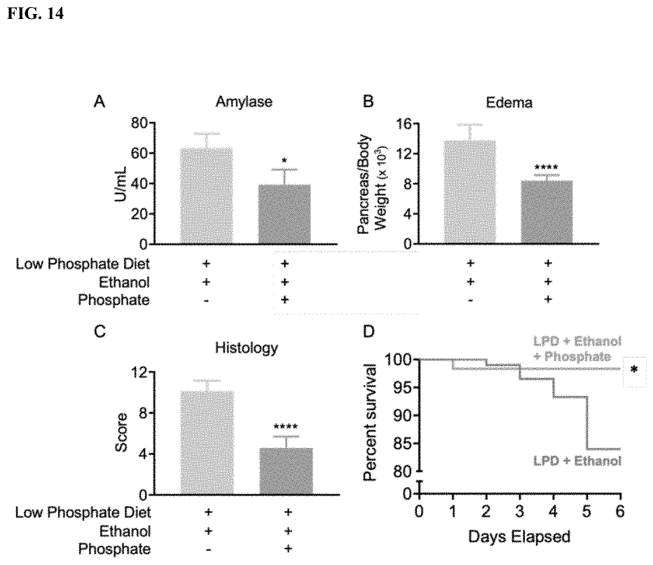

Next, it was further tested as to whether phosphate treatment would protect mice from the ethanol-induced pancreatitis. Mice were fed a low phosphate diet for 3 weeks and then gavaged with ethanol (3 mg/kg) daily for 5 days. In the treatment group, mice were administered sodium phosphate (50 mg/kg) by orogastric gavage daily for 5 days. Twelve hours after the last ethanol dose, animals were euthanized. Serum amylase levels, pancreatic edema, and histological scoring for pancreatitis severity are shown (FIG. 14A-C) (*P<0.05, ****P<0.0001). The survival of mice in this study is shown in FIG. 14D. Phosphate administration significantly reduced mortality of ethanol-treated mice on the low phosphate diet (*P<0.0001, n=17).

These findings indicate that phosphate protects mice against ethanol-induced injury and suggest that phosphate may protect against other causes of pancreatitis.

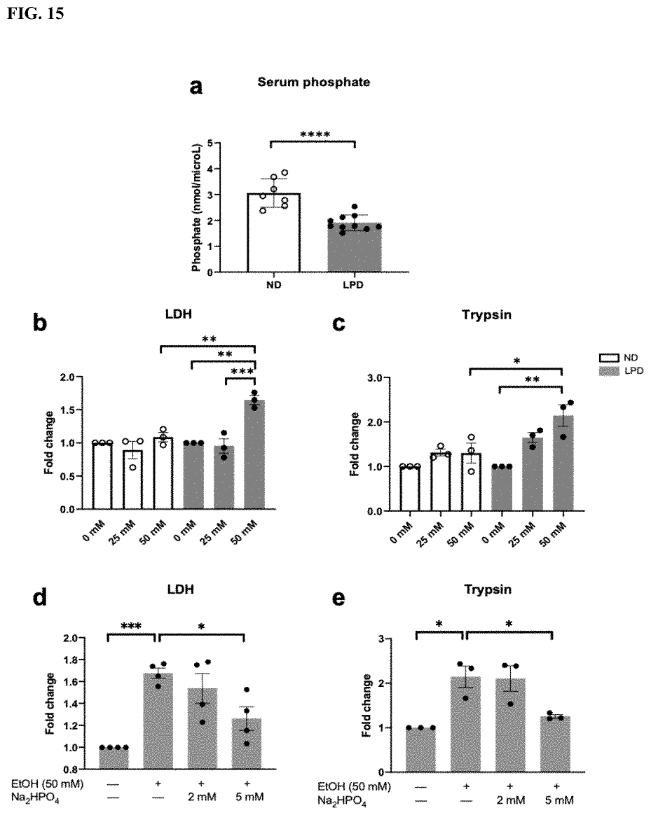

In order to determine the effect of ethanol exposure and phosphate levels on acinar cell function, studies were conducted in vitro. Pancreata was harvested from ND-fed and LPD-fed C57BL/6J mice and isolated acinar cells using phosphate-free buffer solutions. Acinar cell function was assessed at various concentrations of ethanol (0-50 mM). Lactate dehydrogenase (LDH)—a measure of cytotoxicity—was significantly increased in LPD-derived acini exposed to 50 mM ethanol (FIG. 15B). This group also demonstrated significantly increased levels of trypsin activity (FIG. 15C). Notably, the same effects were not seen in pancreatic acini harvested from mice maintained on ND that were exposed to the same concentration of ethanol. These data demonstrate that low phosphate levels exacerbated ethanol-induced acinar cell dysfunction.

Next, it was investigated as to whether phosphate supplementation could preserve pancreatic acinar cell function. Pancreatic acini from LPD-fed mice were isolated in phosphate-free buffer and treated with 50 mM ethanol and 0-5 mM Na₂HPO₄. The addition of 5 mM Na₂HPO₄ significantly decreased LDH (FIG. 15D) and trypsin activity (FIG. 15E) in pancreatic acini exposed to ethanol. These data indicate that the deleterious effects of ethanol are effectively prevented by restoration of normal phosphate levels.

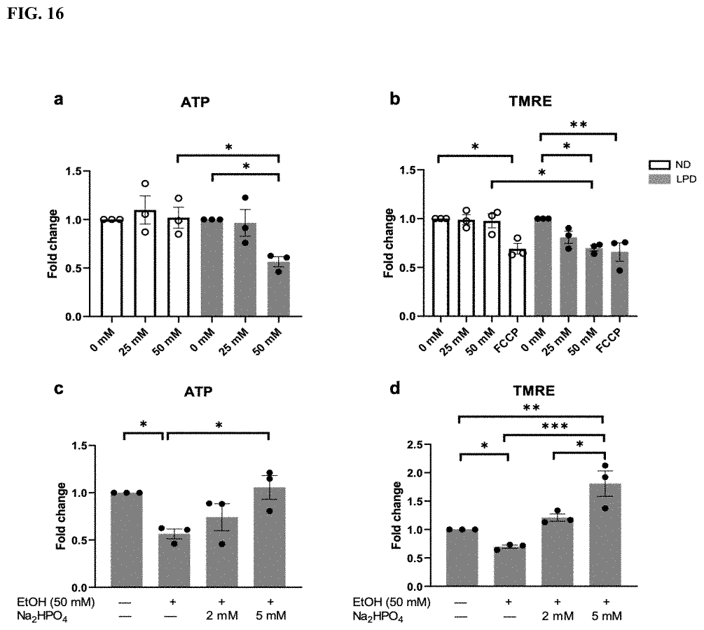

Phosphate level influenced acinar cell susceptibility to ethanol-induced injury by altering cellular ATP stores and mitochondrial function. To further explore the mechanism underlying the effect of phosphate on ethanol-induced acinar cell dysfunction, cellular ATP content and mitochondrial function was investigated. In LPD-derived acini exposed to 50 mM ethanol, total cellular ATP levels were significantly decreased (FIG. 16A), suggesting that reduced energy stores may drive or develop from acinar cell pathology. The same effect was not observed in ND-derived acini, which indicates that chronic phosphate depletion may significantly alter cellular energetics in pancreatic acinar cells.

In order to determine if phosphate sensitizes mitochondria to the effects of alcohol, we measured TMRE fluorescence intensity, a measure of mitochondrial membrane potential that indicates normal mitochondrial function. TMRE fluorescence intensity was significantly decreased in LPD acini stimulated with 50 mM ethanol (FIG. 16B). Compellingly, the loss of mitochondrial activity was similar to that induced by 20 μM FCCP, a potent mitochondrial uncoupler. These data point to mitochondrial dysfunction and altered cellular energetics as potential drivers of increased cell death and increased zymogen activation which lead to the in vivo manifestations of LPD-associated acute alcohol-induced pancreatitis.

This concept is further reinforced by the effect of phosphate supplementation on LPD-derived, ethanol-exposed acinar cell function. Pancreatic acini from LPD-fed mice were again isolated in phosphate-free buffer and treated with 50 mM ethanol and 0-5 mM Na₂HPO₄. In a dose-dependent manner, phosphate supplementation restored acinar cell function with significantly increased ATP levels observed in ethanol-exposed pancreatic acini treated with phosphate (FIG. 16C). TMRE fluorescence intensity was also markedly improved with phosphate supplementation (FIG. 16D). Therefore, normal acinar cell mitochondrial activity was restored by phosphate. These results indicate that phosphate has a significant protective effect on pancreatic tissue on a cellular level.

### Example 4: Nicotinic Compositions for Treatment of Pancreatitis

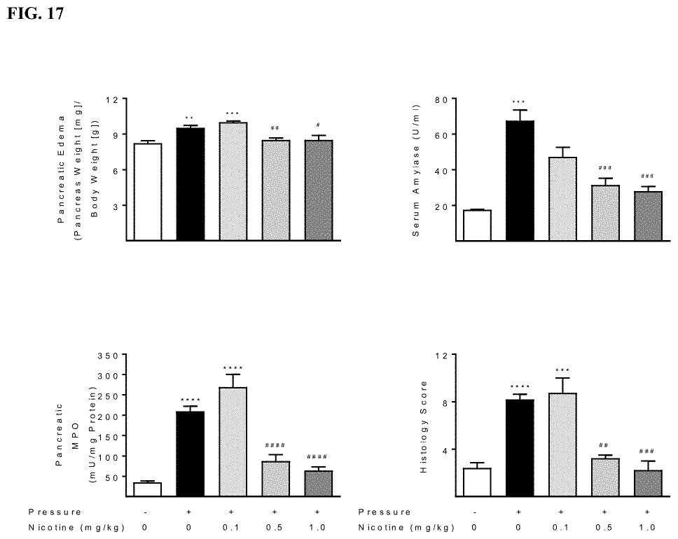

As demonstrated in FIG. 17, nicotine dose-dependently inhibits ERCP pressure-induced pancreatic inflammation, including pancreatic edema, serum amylase, pancreatic MPO levels, and histopathology. **P<0.01 vs. Pressure −/Nicotine 0; ***P<0.001 vs. Pressure −/Nicotine 0; ****P<0.0001 vs. Pressure −/Nicotine 0; #P<0.05 vs. Pressure +/Nicotine 0; ##P<0.01 vs. Pressure +/Nicotine 0; ###P<0.001 vs. Pressure +/Nicotine 0; ####P<0.0001 vs. Pressure +/Nicotine 0. Accordingly, nicotine and related active agents can be used alone, or with phosphate supplementation to treat or protect from pancreatitis.

GTS-21 (DMXBA) is a derivative of the natural product anabaseine that acts as a partial agonist at neural nicotinic acetylcholine receptors. GTS-21 is 3-2,4-dimethoxybenzylidene anabaseine. Experiments further demonstrated that systemic GTS-21 administration significantly protected the pancreas against pressure-induced pancreatitis, and that prior splenectomy abolished this effect of GTS-21. This showed that some type of splenocyte (e.g. T cell) mediates the protective effect of GTS-21. The results demonstrated that splenocytes depleted of T cells do not protect against pressure-induced acute pancreatitis in response to GTS-21 administration, demonstrating that the protective effect of GTS-21 in acute pancreatitis requires splenic T cells.

All references cited herein are fully incorporated by reference, unless explicitly indicated otherwise.

1. A method for treating or preventing pancreatitis in a subject in need thereof, comprising, administering a composition comprising an effective amount of phosphate to the subject so as to treat or prevent pancreatitis in the subject.

2. The method of claim 1, wherein the subject is at risk for developing pancreatitis.

3. The method of claim 2, wherein the subject has a biliary tract disorder.

4. The method of claim 3, wherein the biliary tract disorder is gallstone.

5. The method of claim 2, wherein the subject is an alcohol user.

6. The method of claim 2, wherein the subject has hypertriglyceridemia.

7. The method of claim 2, wherein the subject has hypercalcemia.

8. The method of claim 2, wherein the subject is undergoing treatment with one or more active agents that increase risk of pancreatitis.

9. The method of claim 2, wherein the subject has a genetic predisposition to develop pancreatitis.

10. The method of claim 2, wherein the subject will undergo a post-endoscopic retrograde cholangiopancreatography procedure.

11. The method of claim 2, wherein the subject has an autoimmune disease.

12. The method of any one of claims 1 to 11, wherein the subject has clinical hypophosphatemia.

13. The method of any one of claims 1 to 11, wherein the subject does not have clinical hypophosphatemia.

14. The method of claim 13, wherein the subject has normal phosphate levels or subclinical hypophosphatemia.

15. The method of claim 12 or 13, wherein the subject at risk for developing pancreatitis is tested for serum phosphate levels, and phosphate is administered if the patient does not have hyperphosphatemia.

16. The method of any one of claims 1 to 15, wherein the phosphate comprises sodium phosphate and/or potassium phosphate.

17. The method of claim 16, wherein sodium phosphate is administered intravenously.

18. The method of claim 17, wherein the sodium phosphate is administered a plurality of times.

19. The method of any one of claims 1 to 16, wherein the phosphate is administered orally.

20. The method of claim 19, wherein the phosphate is administered continually as a dietary supplement.

21. The method of any one of claims 1 to 20, wherein the patient is further administered one or more agents selected from: an α7 nicotinic acetylcholine receptor agonist, TRPV4 antagonist, an anticoagulant, pancreatic enzyme inhibitor, magnesium, non-steroidal anti-inflammatory drug (NSAID), calcineurin inhibitor, and calcium channel blocker.

22. The method of claim 21, wherein the agent and is delivered orally or transdermally.

23. The method of claim 22, wherein the nicotinic agonist is delivered by a transdermal patch.

24. The method of claim 1, wherein the subject has pancreatitis.

25. The method of claim 24, wherein the pancreatitis is associated with a biliary tract disorder.

26. The method of claim 25, wherein the biliary tract disorder is gallstone.

27. The method of claim 24, wherein the pancreatitis is associated with alcohol use.

28. The method of claim 24, wherein the pancreatitis is associated with hypertriglyceridemia.

29. The method of claim 24, wherein the pancreatitis is associated with hypercalcemia.

30. The method of claim 24, wherein the pancreatitis is associated with a post-endoscopic retrograde cholangiopancreatography procedure.

31. The method of claim 24, wherein the pancreatitis is associated with an autoimmune disease.

32. The method of claim 24, wherein the pancreatitis is idiopathic pancreatitis.

33. The method of claim 24, wherein the pancreatitis is acute pancreatitis.

34. The method of claim 24, wherein the pancreatitis is chronic pancreatitis.

35. The method of any one of claims 24 to 33, wherein the pancreatitis is mild acute pancreatitis.

36. The method of any one of claims 24 to 33, wherein the pancreatitis is moderately severe pancreatitis.

37. The method of any one of claims 24 to 33, wherein the pancreatitis is severe pancreatitis.

38. The method of any one of claims 24 to 37, wherein the subject has clinical hypophosphatemia.

39. The method of any one of claims 24 to 38, wherein the subject does not have clinical hypophosphatemia.

40. The method of claim 39, wherein the subject has normal phosphate levels or subclinical hypophosphatemia.

41. The method of claim 40, wherein the subject is tested for phosphate levels, and phosphate is administered if the patient does not have hyperphosphatemia.

42. The method of any one of claims 24 to 41, wherein the phosphate comprises sodium phosphate and/or potassium phosphate.

43. The method of claim 42, wherein the sodium phosphate is administered intravenously.

44. The method of claim 43, wherein the sodium phosphate is administered a plurality of times.

45. The method of any one of claims 24 to 44, wherein the phosphate is administered orally.

46. The method of claim 45, wherein the phosphate is administered continually as a dietary supplement.

47. The method of any one of claims 24 to 46, wherein the patient is further administered one or more agents selected from: an α7 nicotinic acetylcholine receptor agonist, TRPV4 antagonist, an anticoagulant, pancreatic enzyme inhibitor, magnesium, non-steroidal anti-inflammatory drug (NSAID), calcineurin inhibitor, and calcium channel blocker.

48. The method of claim 47, wherein the agent is nicotine and is delivered orally or transdermally.

49. The method of claim 48, wherein the nicotinic agonist is delivered by a transdermal patch.

50. A pharmaceutical composition comprising an effective amount of a phosphate salt and one or more of: an α7 nicotinic acetylcholine receptor agonist, TRPV4 antagonist, an anticoagulant, pancreatic enzyme inhibitor, magnesium, non-steroidal anti-inflammatory drug (NSAID), calcineurin inhibitor, and calcium channel blocker.

51. The pharmaceutical composition of claim 50, formulated for intravenous administration.

52. The pharmaceutical composition of claim 50, formulated for intradermal administration.

53. The pharmaceutical composition of claim 50, formulated for oral administration.
